(19) United States
(12) Patent Application Publication
(10) Pub. No.: US 2008/0305060 A1
(43) Pub. Date: Dec. 11, 2008

(54) AQUEOUS-ALCOHOLIC DEPIGMENTING GELS

(75) Inventors: FABIENNE LOUIS, Villeneuve-Loubet (FR); Sandrine Orsoni, Mandelieu (FR); Laurent Fredon, Roquefort Les Pins (FR)

Correspondence Address: BUCHANAN, INGERSOLL & ROONEY PC, POST OFFICE BOX 1404, ALEXANDRIA, VA, 22313-1404, US

(73) Assignee: GALDERMA RESEARCH & DEVELOPMENT, Biot (FR)

(21) Appl. No.: 12/137,384

(22) Filed: Jun. 11, 2008

Related U.S. Application Data
(63) Continuation of application No. PCT/EP2006/069611, filed on Dec. 12, 2006.
(60) Provisional application No. 60/752,593, filed on Dec. 22, 2005.

(30) Foreign Application Priority Data
Dec. 12, 2005 (FR) 0512564
Jun. 21, 2007 (WO) WO 2007/068701 A1

(51) Int. Cl. A61K 8/18; A61P 17/00

(52) U.S. Cl. 424/62

(57) ABSTRACT

Stable, topically applicable cosmetic/pharmaceutical skin depigmenting compositions contain at least one phenolic compound, at least one retinoid and at least one corticoid, and are formulated as aqueous-alcoholic gels in topically applicable, physiologically acceptable media therefor.

### CROSS-REFERENCE TO PRIORITY/PCT/PROVISIONAL APPLICATIONS

This application claims priority under 35 U.S.C. § 119 of FR 0512564, filed Dec. 12, 2005, and of Provisional Application No. 60/752,593, filed Dec. 22, 2005, and is a continuation of PCT/EP 2006/069611, filed Dec. 12, 2006, and designating the United States, published in the English language as WO 2007/068701 A1 on Jun. 21, 2007, each hereby expressly incorporated by reference in its entirety and each assigned to the assignee hereof.

### BACKGROUND OF THE INVENTION

1. Technical Field of the Invention

The present invention relates to depigmenting compositions for cosmetic or pharmaceutical application, comprising a phenolic derivative, a retinoid, especially a dispersed retinoid, and a corticoid, formulated as aqueous-alcoholic gels.

By virtue of its composition, this gel provides the compositions with both stability and harmlessness.

2. Description of Background and/or Related and/or Prior Art

Among the therapeutic agents recommended for the treatment of cutaneous hyperpigmentation, phenolic derivatives such as hydroquinone and derivatives thereof have for decades been among the active agents that are the most effective. The therapeutic indication of these agents results from the observation of cutaneous depigmentation in the case of operatives in the rubber industry, in which certain of these products are used as antioxidants. Subsequently, numerous studies have confirmed their efficacy, alone or combined with other depigmenting agents [Jorge L. Sanchez, M.D. and Miguel Vazquez, M.D., International Journal of Dermatology, January-February 1982 Vol. 21, pp. 55-58]. They are thus found to be active agents that are virtually indispensable in the treatment of hyperpigmentation and are consequently present in many commercial products. Hydroquinone has been the subject of various patent application filings, and in particular U.S. Pat. No. 3,856,934 in which hydroquinone is in combination with retinoic acid and a corticoid, as a depigmenting composition.

However, the incorporation of a phenolic derivative such as hydroquinone presents, inter alia, two major drawbacks.

Firstly, the degradation of formulations containing phenolic derivatives such as hydroquinone, alone or in combination with other active principles, is often observed. Specifically, hydroquinone is known for its sensitivity to oxidation and to heat, resulting in reduced efficacy, rapid browning of the formulations and occasionally even demixing or phase separation of the formulation.

This problem is found to be an obstacle to obtaining compositions comprising hydroquinone with several active agents, especially a phenolic derivative and a retinoid.

In the prior art, sulfite salts are conventionally used to reduce this phenomenon, but they are insufficient to overcome this drawback. They can also impair the viscosity of electrolyte-sensitive formulations and thereby result in sedimentation of the active agents (for example retinoids). Specifically, the carbomers conventionally used to provide a minimum level of viscosity are affected by the electrolytes of sulfite salts and are therefore no longer sufficient alone to allow good stability of the retinoid.

Furthermore, to accelerate their dissolution, phenolic derivatives such as hydroquinone are often exposed to heat during the preparation of the composition, especially in standard emulsions, this phenomenon initiating and accelerating the browning phenomenon.

The second drawback caused by the presence of phenolic derivatives such as hydroquinone, alone or in combination with other active agents, in the composition is their high irritant power.

As a result of its irritant power, hydroquinone at high concentration can give rise to post-inflammatory hypermelanosis and ochronosis phenomena.

Local irritation and dermatitis may develop after a prolonged use of hydroquinone at high concentration [“N-acetyl-4S cysteaminylphenol as a new type of depigmenting agent” Jimbow K., Arch. Dermatol. 1991 October; 127 (10): 1528-1534].

Treatment with hydroquinone may be accompanied by irritation that may lead to a post-inflammatory hyperpigmentation. The incidence of the irritation depends on the hydroquinone concentration. This irritation is relatively high for 10% concentrations and reduces greatly for preparations with a 5% dose, and is considered to be virtually nonexistent at a concentration of 2% [“Les agents chimiques dépigmentants (Depigmenting chemical agents)” JP. Ortonne Ann. Dermatol. Venerol., 1986, 113: 733-736].

The selected galenic form may thus play a predominant role in minimizing these effects.

The existing problem is, thus, that of providing compositions containing a phenolic derivative, a retinoid and a corticoid that are physically stable over time, thus ensuring that the formulation and the active agents remain unchanged. The product must also show good cosmeticity and have little irritant nature.

### SUMMARY OF THE INVENTION

It has now surprisingly been determined that aqueous-alcoholic gels comprising suitable excipients provide good results in terms of physical and chemical stability. Same also offer an excellent compromise from stability, especially to temperature and oxidation, efficacy, harmlessness and cosmeticity.

A novel process has also been developed for preparing the compositions according to the invention, which may be prepared under cold conditions, without heating, thus making it possible to avoid exposing the phenolic derivative to heat.

The present invention thus features depigmenting compositions comprising, formulated into a physiologically acceptable medium, at least one phenolic derivative, a retinoid, especially a dispersed retinoid, and a corticoid, such compositions being aqueous-alcoholic gels.

The term “physiologically acceptable medium” means a medium which is compatible with the skin, the mucous membranes and/or the integuments.

The term “aqueous-alcoholic gel” means an aqueous gel comprising alcohol, and optionally containing a small proportion (up to 15%) of fatty phase.

All proportions are expressed as weight percentages relative to the total weight of the composition.

### DETAILED DESCRIPTION OF BEST MODE AND SPECIFIC/PREFERRED EMBODIMENTS OF THE INVENTION

The compositions according to the invention preferably comprise from 1% to 40% of alcohol.

Among the alcohols that may be included, exemplary are ethanol, isopropanol and butanol and mixtures thereof. The alcohol is preferably ethanol.

The compositions according to the invention may also preferably comprise one or more of the following ingredients:

a) a carbomer,

b) another gelling agent (or a gelling agent different from carbomers),

c) an antioxidant,

d) a chelating agent.

The compositions according to the invention of aqueous-alcoholic gel type offer good skin tolerance. Same are also easier to spread than a viscous emulsion and leaves a pleasant sensation of freshness.

More particularly, the present invention features aqueous-alcohol gels for depigmenting applications, comprising one or more of the following constituents:

from 0.01% to 10% of a phenolic derivative,

from 0.0001% to 5% of a retinoid,

from 0.01 to 5% of a corticoid,

from 0.01% to 10% of carbomer and/or other gelling agents,

from 0.01% to 2% of antioxidants, and

from 0.01% to 1% of chelating agent.

A preferred composition according to the invention comprises:

4.00% of phenolic derivative,

0.10% of retinoid,

0.05% of a corticoid,

from 5% to 20% of ethanol,

from 0.10% to 1% of carbomer,

0.10% to 5% of one or more another gelling agents,

from 0.10% to 0.40% of sulfite salts,

0.10% of EDTA.

Among the carbomers, examples thereof include Carbopol 981, Carbopol 980, Carbopol ETD 2020, Carbopol Ultrez 10 NF marketed by BF Goodrich.

Among the other possible gelling agents, examples thereof include xanthan gum such as Keltrol T marketed by Kelco, acrylate/C10-C30 alkyl acrylate crosspolymer such as the product marketed under the trademark Pemulen TR1 or Carbopol 1382 by BF Goodrich, hydroxypropylcellulose, such as the product marketed under the trademark Natrosol HHX 250 by Aqualon, and acrylamide/sodium acryloyldimethyltaurate copolymer and isohexadecane and polysorbate 80, marketed under the trademark Simulgel 600 by SEPPIC.

Among the antioxidants, examples thereof include ascorbic acid and its salts, tocopherols and sulfite salts such as sodium metabisulfite or sodium sulfite.

Examples of chelating agents include ethylenediaminetetraacetic acid (EDTA), calcium disodium edetate and sodium edetate.

Phenolic derivatives that are exemplary include hydroquinone, 4-hydroxyanisole, hydroquinone monoethyl ether and hydroquinone monobenzyl ether. Preferably hydroquinone is employed.

The term “retinoid” means any compound that binds to the retinoic acid receptors (RARs) and/or to retinoic X receptors (RXRs), and also precursors and derivatives thereof.

Preferably, the retinoid is a compound selected from the family of benzonaphthalene-based retinoids as described in EP-0, 199.636. Adapalene (6-[3-(1-adamantyl)-4-methoxyphenyl]-2-naphthoic acid) and precursors and/or derivatives thereof will be preferred in particular. Tretinoin and isotretinoin may also be employed.

The term “retinoid precursors” means the immediate biological precursors or substrates thereof, and also chemical precursors thereof.

The term “retinoid derivatives” means both the metabolic derivatives thereof and the chemical derivatives thereof.

The term “corticoid” means clobetasone butyrate, clobetasol propionate, clobetasol dipropionate, hydrocortisone, cortisone, prednisolone, miconazole, prednisone, triamcinolone acetonide, methylprednisolone, fluometholone, fluocinolone acetonide, desonide, betamethasone, dexamethasone, aclosone or mixtures thereof.

Particularly, the compositions according to the invention comprise, as corticoid, desonide or fluocinolone acetonide.

Of course, the amount of the active agents in the composition according to the invention will depend on the selected combination and thus particularly on the quality of the desired treatment.

The subject compositions may also comprise additives usually employed in cosmetics or pharmaceuticals, such as a neutralizer, a humectant and/or co-solvent, an emollient, a calmative, a physical sunblock or a chemical sunscreen, a preservative or a pH corrector, or mixtures thereof.

Needless to say, one skilled in this art will take care to select this or these additional compound(s), and/or the amount thereof, such that the advantageous properties of the composition according to the invention are not, or are not substantially, adversely affected.

The composition may preferably comprise a chemical sunscreen or a physical sunblock.

These additives may be present in the composition in a proportion of from 0.001% to 20% by weight relative to the total weight of the composition.

Examples of neutralizers include an amine base such as triethanolamine, diethanolamine or tromethamine.

An example of a pH corrector is citric acid. Examples of humectants and/or co-solvents include glycerol, sorbitol, propylene glycol and macrogol 400.

The compositions according to the invention may also comprise a fatty phase in a proportion ranging from 0.01% to 15%, comprising essentially an emollient. Examples of such emollients include a mineral oil such as Primol 352, Marcol 82, Marcol 152 and Marcol 352 marketed by Esso; a plant oil such as sweet almond oil, palm oil, soybean oil, sesame seed oil, sunflower oil, an ester such as cetearyl isononanoate, for instance the product marketed under the trademark Cetiol SN by Cognis France, diisopropyl adipate, for instance the product marketed under the trademark Ceraphyl 230 by ISP, isopropyl palmitate, for instance the product marketed under the trademark Crodamol IPP by Croda, caprylic/capric triglyceride, such as Miglyol 812 marketed by Hüls/Lambert Rivière; a silicone oil such as a dimethicone, for instance the product marketed under the trademark Q7 9120 by Dow Corning, or a cyclomethicone, for instance the product marketed under the trademark Mirasil CM5 by SACI-CFPA.

Examples of calmatives include allantoin and talc.

Examples of sunscreens include physical sunblocks such as titanium dioxide and chemical sunscreens such as octocrylene, ethylhexyl methoxycinnamate.

Examples of preservatives include benzalkonium chloride, phenoxyethanol, benzyl alcohol, diazolidinylurea and parabens, or mixtures thereof.

The present invention also features administration of the compositions as described above, as medicinal products.

This invention also features a process, performed at room temperature, for preparing a composition of aqueous-alcoholic gel type, comprising the following steps:

a) the preparation of an aqueous formulation phase comprising the mixture of at least one gelling agent with water until the mixture is totally homogeneous. This phase comprises water, the chelating agent, and the gelling agents;

b) the preparation of a first active phase comprising the mixture of the phenolic derivative with the alcohol, which is stirred until dissolution is complete;

c) the preparation of a second active phase comprising the mixture of the retinoid with at least one humectant, which is stirred until a smooth, homogeneous dispersion is obtained;

d) the preparation of a third active phase comprising the mixture of the corticoid with at least one humectant until a homogeneous dispersion is obtained. The corticoid may be dispersed in a humectant which is identical or different from the humectant used at step c).

e) the mixing of the various active phases obtained in b), c) and d) into the formulation phase obtained in a) independently, with stirring until fully incorporated.

The term “ambient temperature” means a temperature ranging from 20 to 30° C., preferably from 23° C. to 27° C., preferably equal to 25° C.

Alternatively, the retinoid and the corticoid may be directly independently introduced with the active phase obtained in b) into the formulation phase obtained in a).

The monitoring of the native pH of the mixture and the correction, where necessary, with a solution of a neutralizer, and the incorporation of the optional additives may be performed, depending on their chemical nature, during one of the steps of the preparation process described above.

Thus, in one particular embodiment of the process according to the present invention, at least one neutralizing agent may be introduced into the formulation phase after step a).

In one particular embodiment of the process according to the present invention, at least one antioxidant predissolved in water may be introduced into the formulation phase after step a).

In a last particular embodiment of the process of the invention, a fatty phase is introduced into the gel obtained after step e), by reason of realization of an emulsification at a high speed.

The expression “formulation phase” means the mixture of a group of ingredients different from the active agents introduced together into a single phase.

The term “active phase” means a formulation phase containing one or more active agents.

The present invention also features the application, whether regime or regimen, of the novel compositions as described above in cosmetics and dermatology.

The compositions of the invention are particularly suitable for treating and/or preventing dermatological complaints, conditions or afflictions associated with pigmentation disorders such as melasma, chloasma, lentigines, senile lentigo, vitiligo, freckles, post-inflammatory hyperpigmentations caused by an abrasion and/or a burn and/or a scar and/or a dermatosis and/or a contact allergy; nevi, genetically determined hyperpigmentation, hyperpigmentation of metabolic or medicational origin, melanomas or any other hyperpigmentary lesions.

The compositions according to the invention also find an application in cosmetics, in particular for preventing and/or combating the harmful effects of sunlight and/or for combating photo-induced or chronological aging of the skin and the integuments.

The compositions according to the invention also find application in body and hair hygiene.

In order to further illustrate the present invention and the advantages thereof, the following specific examples are given, it being understood that same are intended only as illustrative and in nowise limitative. In said examples to follow, all parts and percentages are given by weight, unless otherwise indicated.

### FORMULATION EXAMPLES

In the compositions below (Examples 1 to 11), the proportions of the various constituents are expressed as weight percentages relative to the total weight of the composition.

### Example 1

| Starting materials | % |
|---|---|
| Hydroquinone | 4.00 |
| Adapalene | 0.10 |
| Desonide | 0.05 |
| Ethanol | 20.00 |
| EDTA | 0.10 |
| Carbopol 980 | 0.40 |
| Acrylate/C10-C30 alkyl acrylate crosspolymer | 0.40 |
| Sodium metabisulfite | 0.20 |
| Sodium sulfite | 0.20 |
| Propylene glycol | 5.00 |
| Glycerol | 5.00 |
| Phenoxyethanol | 1.00 |
| Aqueous 10% tromethamine solution | (Qs pH 5.5-6.5) |
| Purified water | qs 100 |

### Example 2

| Starting materials | % |
|---|---|
| Hydroquinone | 4.00 |
| Adapalene | 0.10 |
| Desonide | 0.05 |
| Ethanol | 20.00 |
| EDTA | 0.10 |
| Carbopol 980 | 0.40 |
| Acrylate/C10-C30 alkyl acrylate crosspolymer | 0.40 |
| Sodium metabisulfite | 0.20 |
| Sodium sulfite | 0.20 |
| PEG400 | 10.00 |
| Phenoxyethanol | 1.00 |
| Aqueous 10% tromethamine solution | (qs pH 5.5-6.5) |
| Purified water | qs 100 |

### Example 3

| Starting materials | % |
|---|---|
| Hydroquinone | 4.00 |
| Adapalene | 0.10 |
| Desonide | 0.05 |
| Ethanol | 20.00 |
| EDTA | 0.10 |
| Carbopol 980 | 0.40 |
| Acrylate/C10-C30 alkyl acrylate crosspolymer | 0.60 |
| Sodium metabisulfite | 0.20 |
| Sodium sulfite | 0.20 |
| Propylene glycol | 5.00 |
| Glycerol | 5.00 |
| Phenoxyethanol | 1.00 |
| Aqueous 10% tromethamine solution | 4.00 |
| Citric acid | (qs pH 5-7) |
| Purified water | qs 100 |

### Example 4

| Starting materials | % |
|---|---|
| Hydroquinone | 4.00 |
| Adapalene | 0.10 |
| Desonide | 0.05 |
| Ethanol | 20.00 |
| EDTA | 0.10 |
| Carbopol 980 | 0.30 |
| Carbopol 981 | 0.30 |
| Xanthan gum | 0.40 |
| Sodium metabisulfite | 0.20 |
| Sodium sulfite | 0.20 |
| Phenoxyethanol | 1.00 |
| Propylene glycol | 5.00 |
| Glycerol | 5.00 |
| Aqueous 10% tromethamine solution | 4.00 |
| Citric acid | (qs pH 5-7) |
| Purified water | qs 100 |

### Example 5

| Starting materials | % |
|---|---|
| 4-Hydroxyanisole | 4.00 |
| Adapalene | 0.10 |
| Fluocinolone acetonide | 0.01 |
| Ethanol | 15.00 |
| EDTA | 0.10 |
| Carbopol 980 | 0.60 |
| Xanthan gum | 0.40 |
| Sodium metabisulfite | 0.20 |
| Sodium sulfite | 0.20 |
| Phenoxyethanol | 1.00 |
| Propylene glycol | 5.00 |
| Glycerol | 5.00 |
| Aqueous 10% tromethamine solution | 4.00 |
| Citric acid | (qs pH 5-7) |
| Purified water | qs 100 |

### Example 6

| Starting materials | % |
|---|---|
| 4-Hydroxyanisole | 4.00 |
| Adapalene | 0.10 |
| Desonide | 0.05 |
| Ethanol | 20.00 |
| EDTA | 0.10 |
| Carbopol 980 | 0.40 |
| Acrylate/C10 C30 Alkyl Acrylate crosspolymer | 0.40 |
| Sodium metabisulfite | 0.20 |
| Sodium sulfite | 0.20 |
| Phenoxyethanol | 1.00 |
| Propylene glycol | 5.00 |
| Glycerol | 5.00 |
| Aqueous 10% tromethamine solution | (qs pH 5.5-6.5) |
| Purified water | qs 100 |

### Example 7

| Starting materials | % |
|---|---|
| Hydroquinone | 2.00 |
| Tretinoin | 0.10 |
| Aclosone | 0.01 |
| Ethanol | 30.00 |
| Sodium edetate | 0.10 |
| Carbopol 981 | 0.50 |
| Carbopol 1382 | 0.50 |
| Sodium metabisulfite | 0.20 |
| Sodium sulfite | 0.20 |
| Propylene glycol | 5.00 |
| Glycerol | 5.00 |
| Aqueous 10% tromethamine solution | (qs pH 5-7) |
| Purified water | qs 100 |

### Example 8

| Starting materials | % |
|---|---|
| 4-Hydroxyanisole | 5.00 |
| Tretinoin | 0.10 |
| Desonide | 0.05 |
| Ethanol | 15.00 |
| Calcium disodium edetate | 0.10 |
| Carbopol ETD 2020 | 0.40 |
| Hydroxypropylcellulose | 1.00 |
| Sodium metabisulfite | 0.20 |
| Sodium sulfite | 0.20 |
| Propylene glycol | 5.00 |
| Macrogol E400 | 5.00 |
| Aqueous 10% tromethamine solution | 4.00 |
| Citric acid | (qs pH 5-7) |
| Purified water | qs 100 |

### Example 9

| Starting materials | % |
|---|---|
| Hydroquinone | 4.00 |
| Adapalene | 0.10 |
| Desonide | 0.05 |
| Ethanol | 20.00 |
| EDTA | 0.10 |
| Carbopol 981 | 0.60 |
| Xanthan gum | 0.40 |
| Sodium metabisulfite | 0.20 |
| Sodium sulfite | 0.20 |
| Liquid paraffin | 10.00 |
| Phenoxyethanol | 1.00 |
| Propylene glycol | 5.00 |
| Glycerol | 5.00 |
| Aqueous 10% tromethamine solution | 4.00 |
| Citric acid | (qsp pH 5-7) |
| Purified water | Qsp 100 |

### Example 10

| Starting materials | % |
|---|---|
| 4-Hydroxyanisole | 4.00 |
| Adapalene | 0.10 |
| Desonide | 0.05 |
| Ethanol | 20.00 |
| EDTA | 0.10 |
| Carbopol 980 | 0.40 |
| Acrylate/C10 C30 Alkyl Acrylate crosspolymer | 0.40 |
| Sodium metabilsulfite | 0.20 |
| Sodium sulfite | 0.20 |
| PEG 400 | 10.00 |
| Phenoxyethanol | 1.00 |
| Aqueous 10% tromethamine solution | (qsp pH 5.5-6.5) |
| Purified water | Qsp 100 |

### Example 11

| Starting materials | % |
|---|---|
| 4-hydroxyanisole | 2.00 |
| Adapalene | 0.10 |
| Desonide | 0.05 |
| Ethanol | 5.00 |
| EDTA | 0.10 |
| Carbopol 981 | 0.20 |
| Carbomer 1382 | 0.60 |
| Xanthan gum | 0.40 |
| Sodium metabisulfite | 0.05 |
| Sodium sulfite | 0.05 |
| Liquid paraffin | 10.00 |
| Propylene glycol | 5.00 |
| Glycerol | 5.00 |
| Methyl paraben | 0.20 |
| Allantoin | 0.20 |
| Butylhydroxytoluene | 0.10 |
| Sorbitan monooleate | 1.00 |
| Poloxamer 124 | 0.20 |
| Aqueous 10% tromethamine solution | (qsp pH 4.5) |
| Purified water | Qsp 100 |

The formulations of Examples 1 to 11 may be applied once or twice a day until total depigmentation is achieved, for the treatment of lentigines, chloasma or melasma.

### Example 12

### Stability Study

The compositions according to the invention and a control gel are subjected to various temperatures, and a physical evaluation (color and physical structure of the gel) is carried out over time.

| | |
|---|---|
| Stabilities Example 1 | 45° C.: 1 month, no comments* |
| | 4° C.: 3 months, no comments |
| | Room temperature: 1 year, no comments |

*no comments: no change in the original color and physical structure maintained

| | |
|---|---|
| Stabilities Example 2 | 45° C.: 7 weeks, no comments |
| | 4° C.: 7 weeks, no comments |
| | Room temperature: 1 year, no comments |

| | |
|---|---|
| Stabilities Example 3 | 45° C.: 2 months, no comments |
| | 4° C.: 3 months, no comments |
| | Room temperature: 2.5 months, no comments |

| | |
|---|---|
| Stabilities Example 5 | 45° C.: 2.5 months, no comments |
| | 4° C.: 2.5 months, no comments |
| | Room temperature: 1 year, no comments |

| | |
|---|---|
| Stabilities Example 4 | 45° C.: 2 months, no comments |
| | 4° C.: 3 months, no comments |
| | Room temperature: 2.5 months, no comments |

| | |
|---|---|
| Stabilities Example 8 | 45° C.: 2 months, no comments |
| | 4° C.: 3 months, no comments |
| | Room temperature: 2.5 months, no comments |

| | |
|---|---|
| Stabilities Example 9 | 45° C.: 3 months, no comments |
| | 4° C.: 3 months, no comments |
| | Room temperature: 1 year, no comments |

Control Gel:

| Starting materials | % |
|---|---|
| Hydroquinone | 4.00 |
| Adapalene | 0.10 |
| Desonide | 0.05 |
| EDTA | 0.20 |
| Carbomer | 0.50 |
| Sodium metabisulfite | 0.20 |
| Sodium sulfite | 0.20 |
| Allantoin | 0.20 |
| Methyl paraben | 0.15 |
| Propylene glycol | 15.00 |
| Glycerol | 5.00 |
| Aqueous 10% sodium hydroxide solution | 2.00 |
| Citric acid | (qs pH 5-7) |

| | |
|---|---|
| Stabilities of the | 45° C.: 2 weeks beige, 5 weeks shaded brown, |
| control gel | 1.5 months start of demixing** |
| | 4° C.: 3 months: fluidization** |
| | Room temperature: 1 month fluidization** |

**modification of the physical structure

The stability monitoring of the tests performed shows that the compositions according to the invention are stable over time and at all the temperatures tested, as regards both the color and the physical structure, in contrast with the control gel not in accordance with the invention. The phenol derivative, the retinoid and the corticoid used show no sign of recrystallization.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A stable, topically applicable cosmetic/pharmaceutical skin depigmenting composition comprising a combination depigmentation effective amount of a phenolic compound, a retinoid and a corticoid, formulated as an aqueous-alcoholic gel in a topically applicable, physiologically acceptable medium therefor.

2. The aqueous-alcoholic gel skin depigmenting composition as defined by claim 1, comprising from 1% to 40% of alcohol.

3. The aqueous-alcoholic gel skin depigmenting composition as defined by claim 2, wherein the alcohol comprises ethanol.

4. The aqueous-alcoholic gel skin depigmenting composition as defined by claim 1, further comprising one or more of the following constituents:
a) a carbomer,
b) another gelling agent,
c) an antioxidant,
d) a chelating agent.

5. The aqueous-alcoholic gel skin depigmenting composition as defined by claim 1, comprising:
4.00% of phenolic compound,
0.10% of retinoid,
0.05% of corticoid,
from 5% to 20.00% of ethanol,
from 0.10% to 1% of carbomer,
from 0.10% to 5% of one or more other gelling agents,
from 0.10% to 0.40% of sulfite salts,
0.10% of EDTA.

6. The aqueous-alcoholic gel skin depigmenting composition as defined by claim 1, wherein the phenolic compound comprises hydroquinone.

7. The aqueous-alcoholic gel skin depigmenting composition as defined by claim 1, wherein the retinoid comprises adapalene.

8. The aqueous-alcoholic gel skin depigmenting composition as defined by claim 1, wherein the corticoid is selected from the group consisting of desonide and fluocinolone acetonide.

9. The aqueous-alcoholic gel skin depigmenting composition as defined by claim 1, further comprising a chemical sunscreen or a physical sunblock.

10. A process for preparing the aqueous-alcoholic gel skin depigmenting composition as defined by claim 1, comprising the following steps, conducted at about room temperature:
a) preparing an aqueous formulation phase comprising the mixture of at least one gelling agent with water until the mixture is homogeneous;
b) preparing a first active phase comprising the mixture of the phenolic compound with the alcohol, which is stirred until dissolution is complete;
c) preparing a second active phase comprising the mixture of the retinoid with at least one humectant, which is stirred until a homogeneous dispersion is obtained;
d) preparing a third active phase comprising the mixture of the corticoid with at least one humectant until a homogeneous dispersion is obtained;
e) mixing of the various active phases obtained in b), c) and d) into the formulation phase obtained in a) independently, with stirring until fully incorporated.

11. The process as defined by claim 10, wherein at least one neutralizing agent is introduced into the formulation phase after step a).

12. The process as defined by claim 10, wherein at least one antioxidant pre-dissolved in water is introduced into the formulation phase after step a).

13. The process as defined by claim 10, wherein a fatty phase is introduced into the gel obtained after step (e).

14. A regime or regimen for treating and/or preventing dermatological complaints, conditions or afflictions associated with pigmentation disorders, comprising topically applying onto the skin of an individual in need of such treatment, a thus effective amount of the aqueous-alcoholic gel skin depigmenting composition as defined by claim 1.

15. A regime or regimen for preventing and/or combating the harmful effects of sunlight and/or for combating photo-induced or chronological aging, comprising topically applying onto the skin of an individual in need of such treatment, a thus effective amount of the aqueous-alcoholic gel skin depigmenting composition as defined by claim 1.

16. A regime or regimen for the treatment of lentigines, chloasma or melasma, comprising topically applying onto the skin of an individual in need of such treatment, a thus effective amount of the aqueous-alcoholic gel skin depigmenting composition as defined by claim 1.

17. The aqueous-alcoholic gel skin depigmenting composition as defined by claim 1, wherein the corticoid is selected from the group consisting of clobetasone butyrate, clobetasol propionate, clobetasol dipropionate, hydrocortisone, cortisone, prednisolone, miconazole, prednisone, triamcinolone acetonide, methylprednisolone, fluometholone, fluocinolone acetonide, desonide, betamethasone, dexamethasone, aclosone and mixtures thereof.
